(19) United States
(12) Patent Application Publication
(10) Pub. No.: US 2005/0273111 A1
(43) Pub. Date: Dec. 8, 2005

(54) Methods and apparatus for intervertebral disc removal and endplate preparation

(76) Inventors: Bret A. Ferree, Cincinnati, OH (US); David Tompkins, Milford, OH (US)

Correspondence Address: John G. Posa;Gifford, Krass, Groh, Sprinkle,, Anderson & Citkowski, P.C., PO Box 7021, Troy, MI, 48007, US

(21) Appl. No.: 11/131,999

(22) Filed: May 18, 2005

Related U.S. Application Data
(63) Continuation-in-part of application No. 10/892,795, filed on Jul. 16, 2004.
Application No. 10/892,795 is a continuation-in-part of application No. 10/303,385, filed on Nov. 25, 2002.
Application No. 10/303,385 is a continuation-in-part of application No. 10/191,639, filed on Jul. 9, 2002.
Application No. 10/191,639 is a continuation-in-part of application No. 09/415,382, filed on Oct. 8, 1999, now Pat. No. 6,419,704.
Application No. 10/191,639 is a continuation-in-part of application No. 09/580,231, filed on May 26, 2000, now Pat. No. 6,494,883.
(60) Provisional application No. 60/572,020, filed on May 18, 2004.
(60) Provisional application No. 60/589,752, filed on Jul. 21, 2004.

(51) Int. Cl. A61B 17/16

(52) U.S. Cl. 606/84; 606/167; 606/79; 606/80

(57) ABSTRACT

Improved techniques are disclosed for preparing a disc space to accept an intradiscal device. Methods and apparatus are described to quickly remove disc tissue and improve the surface contact between intradiscal devices and the vertebral end plates (VEP)s. The invention also anticipates the use of navigational devices and CNC controlled machines or robotic arms in conjunction with the disclosed methods and apparatus. The various instruments may be used to remove disc material and/or shape the VEPs. Kits may be supplied with various sizes and shapes of the devices to accommodate discs of different sizes and shapes.

### REFERENCE TO RELATED APPLICATIONS

This application claims priority to U.S. Provisional Patent Application Ser. Nos. 60/572,020, filed May 18, 2004 and 60/589,752, filed Jul. 21, 2004. This application is also a continuation-in-part of U.S. patent application Ser. No. 10/892,795, filed Jul. 16, 2004, which is a continuation-in-part of U.S. patent application Ser. No. 10/303,385, filed Nov. 25, 2002; which is a continuation-in-part of U.S. patent application Ser. No. 10/191,639, filed Jul. 9, 2002; which is a continuation-in-part of U.S. patent application Ser. No. 09/415,382, filed Oct. 8, 1999, now U.S. Pat. No. 6,419,704, and Ser. No. 09/580,231, filed May 26, 2000, now U.S. Pat. No. 6,494,883. The entire content of each application and patent is incorporated herein by reference.

### FIELD OF THE INVENTION

This invention relates generally to surgical procedures and, in particular, to improved methods and apparatus for intervertebral disc removal and endplate preparation.

### BACKGROUND OF THE INVENTION

Current surgical treatments of disc degeneration are destructive. One group of procedures removes the nucleus or a portion of the nucleus; lumbar discectomy falls in this category. A second group of procedures destroy nuclear material; chymopapin (an enzyme) injection, laser discectomy, and thermal therapy (heat treatment to denature proteins) fall in this category. A third group, spinal fusion procedures, either remove the disc or the disc's function by connecting two or more vertebra together with bone.

These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. The insertion of intradiscal devices, such as artificial disc replacements (ADRs) and spinal cages, requires removal of disc material and shaping the vertebral endplates (VEPs) to accept the devices. Prior-art instruments and techniques are not efficient. Removal of the disc material with instruments such as scalpels, curettes, forceps, rasps, and scrapers is a slow, multi-step process. Often surgeons remove only a small piece of disc, for example less than five percent of the disc, each time they withdraw an instrument from the disc space.

The irregular VEPs are shaped after disc removal to improve the surface contact with the intradiscal device. Prior-art techniques include the “free hand” use of burs, chisels, and rasps. Cylindrical guides are also used with cylindrical drills and reamers. Existing cylindrical devices create cylindrical spaces between the vertebrae. FIG. 2A is a lateral view of a prior-art cylindrical guide. The projections from the left of the guide are forced into the disc. FIG. 2B is a view of the end of the device drawn in FIG. 2A. FIG. 2C is a view of the top of the device drawn in FIG. 2A. FIG. 2D is a view of the top of the device drawn in FIG. 2C and the tip of a reamer. The cylindrical reamer has been inserted into the cylindrical guide. FIG. 2E is a lateral view of the spine and the guide drawn in FIG. 2A. The dotted lines indicate the preferred course of the reamer. The guide helps surgeons create a cylindrical hole between the vertebrae. Ideally, surgeons remove and equal amount of bone from each vertebra.

FIG. 2F is a lateral view of the spine and the guide drawn in FIG. 2E. The drawing demonstrates one of the problems of the prior-art device. The guide, if improperly aligned, allows removal of more bone from one vertebra than the other vertebra. Asymmetric bone removal leads to the insertion of a miss-aligned intradiscal device. The intradiscal device may also tilt with time as the device sinks, or subsides, into the exposed softer bone of one of the vertebrae.

### SUMMARY OF THE INVENTION

The present invention improves upon prior art techniques of preparing a disc space to accept an intradiscal device. The invention includes methods and apparatus to quickly remove disc tissue. The invention also includes methods and apparatus to improve the surface contact between intradiscal devices and the vertebral end plates (VEPs). The invention also anticipates the use of navigational devices and CNC controlled machines or robotic arms in conjunction with the disclosed methods and apparatus. The various instruments may be used to remove disc material and/or shape the VEPs. Kits may be supplied with various sizes and shapes of the devices to accommodate discs of different sizes and shapes.

Apparatus directed to the efficient removal of disc material in conjunction with spinal surgery comprises, according to the invention, an evacuator having one or more prongs used to cut disc material and/or shave disc material from a vertebral endplate. The evacuator and other inventive instruments are preferably configured for attachment to a power tool, which may be controlled by a computer-directed navigation or other machine. The evacuator includes a pair of plates, each with prongs, and either or both of which is moveable.

The plates oscillate from side to side in the preferred embodiment. The evacuator could also oscillate towards and away from the power tool, up and down, or in a combination of the above, such as a circular motion. Indeed, instruments according to the invention may oscillate or vibrate in a left to right, cephalad to caudal, or anterior to posterior direction. Alternative combinations of these and other motions may alternatively be used, depending upon the application and desired effect. For example, the tools may reciprocate, rotate, or use random or orbital motions. Tools according to the invention may be driven by ultrasonic vibrations. The entire blade of the instrument may move uniformly. Alternatively, the tip of the blade may move through a greater range or arc of motion than the end of the blade that is attached to the power tool. The end of the blades that attach to the power tools may be configured to cooperate with current or future power tools.

Alternative apparatus directed to the efficient removal of disc material in conjunction with spinal surgery comprises a guide configured for insertion into an intradiscal space and a cutter that fits into the guide for a controlled removal of disc material. In conjunction with a disc replacement device having one or more keels, the apparatus preferably comprises a body having one or more guides to remove vertebral material corresponding to at least one of the keels.

### BRIEF DESCRIPTION OF THE DRAWINGS

FIG. 1A is the view of the top of a disc evacuator of the present invention;

FIG. 1B is a lateral view of the device drawn in FIG. 1A;

FIG. 1C is a lateral view of an alternative embodiment, wherein the top and bottom edges of the device are circular in shape;

FIG. 1D is an axial cross section of the disc and the device drawn in FIG. 1A;

FIG. 1E is a coronal cross section of the cutting portion of the evacuator drawn in FIG. 1A;

FIG. 1F is a coronal cross section of the device drawn in FIG. 1A;

FIG. 1G is the view of an alternative embodiment wherein the evacuator uses a pair of cutting components;

FIG. 2A is a lateral view of a prior-art cylindrical guide;

FIG. 2B is a view of the end of the device drawn in FIG. 2A;

FIG. 2C is a view of the top of the device drawn in FIG. 2A;

FIG. 2D is a view of the top of the device drawn in FIG. 2C and the tip of a reamer;

FIG. 2E is a lateral view of the spine and the guide drawn in FIG. 2A;

FIG. 2F is a lateral view of the spine and the guide drawn in FIG. 2E;

FIG. 3A is a lateral view of the spine and a novel milling guide;

FIG. 3B is a view of the front of the device drawn in FIG. 3A;

FIG. 3C is a view of the front of the cutting guide drawn in FIG. 3B and a cross section of a cutting tool;

FIG. 3D is a view of the front of the device drawn in FIG. 3C and a cross section of a cutting tool;

FIG. 3E is a view of the top of the cutting drawn in FIG. 3A and the cutting tool;

FIG. 3F is a view of the embodiment of the invention drawn in FIG. 3E;

FIG. 3G is a view of an alternative embodiment of a cutting guide and the cutting tool;

FIG. 3H is a lateral view of the end of the cutting tool drawn in FIG. 3F;

FIG. 3I is a view of the end of the cutting tool drawn in FIG. 3H;

FIG. 3J is a view of the end of an alternative embodiment of a cutting tool;

FIG. 3K is view of the end of the cutting tool drawn in FIG. 3J;

FIG. 3L is an anterior view of the spine and a cross section of the cutting tool drawn in FIG. 3K;

FIG. 3M is a lateral view of the spine and the cutting tool drawn in FIG. 3L;

FIG. 4A is a lateral view of the spine and a distraction device;

FIG. 4B is a lateral view of the spine, the distraction device, and the cutting guide drawn in FIG. 3G;

FIG. 4C is an anterior view of the spine and the distraction device drawn in FIG. 4A;

FIG. 4D is an anterior view of the spine, the distraction device, with the cutting guide of FIG. 4B placed over the distraction device;

FIG. 5A is coronal cross section through an alternative embodiment of the invention;

FIG. 5B is a lateral view of the spine and the embodiment of the invention drawn in FIG. 5A;

FIG. 5C is an axial view of the disc and the embodiment of the cutting guide drawn in FIG. 5A;

FIG. 6A is a lateral view of a novel distraction and drill guide;

FIG. 6B is an anterior view of the distracter drawn in FIG. 6A;

FIG. 6C is a lateral view of the spine, the distracter drawn in FIG. 6A, and a drill;

FIG. 6D is an anterior view of an ADR with novel keels that fit into the holes created by the guide and drill drawn in FIG. 6C;

FIG. 6E is an anterior view of the spine;

FIG. 7A is a lateral view of the spine and a novel pressure transducer placed into the disc space after evacuating the disc and possibly after shaping the VEPs;

FIG. 7B is a lateral view of the spine, the transducer drawn in FIG. 7A, and a monitor;

FIG. 7C is a sagittal cross section of the transducer drawn in FIG. 7A;

FIG. 8A is an anterior view of an alternative embodiment of the guide drawn in FIG. 3B;

FIG. 8B is an axial cross section of the guide drawn in FIG. 8A;

FIG. 9A is the view of the top of a blade designed for use with a power tool;

FIG. 9B is a view of the end of the cutting tool drawn in FIG. 9A;

FIG. 9C is a view of the top of an alternative cutting bit;

FIG. 9D is a view of the end of the cutting tool drawn in FIG. 9C;

FIG. 9E is a view of the top of an alternative cutting bit;

FIG. 9F is a view of the end of the cutting tool drawn in FIG. 9E;

FIG. 9G is a view of the top of an alternative cutting bit;

FIG. 9H is a view of the end of the cutting tool drawn in FIG. 9G;

FIG. 9I is a view of the side of an alternative cutting tool;

FIG. 10A is an anterior view of an alternative embodiment of the invention drawn in FIG. 6B;

FIG. 10B is an anterior view of the spine after creating holes with the invention taught in FIG. 10A;

FIG. 10C is an anterior view of an alternative embodiment of the invention drawn in FIG. 6D;

FIG. 11A is an anterior view of an alternative embodiment of the invention drawn in FIG. 10A;

FIG. 11B is a lateral view of the spine, the embodiment of the invention drawn in FIG. 11A;

FIG. 11C is a lateral view of the spine and two K-wires;

FIG. 11D is an end view of a cannulated chisel; and

FIG. 11E is an oblique view of the embodiment of the invention drawn in FIG. 11D.

### DETAILED DESCRIPTION OF THE INVENTION

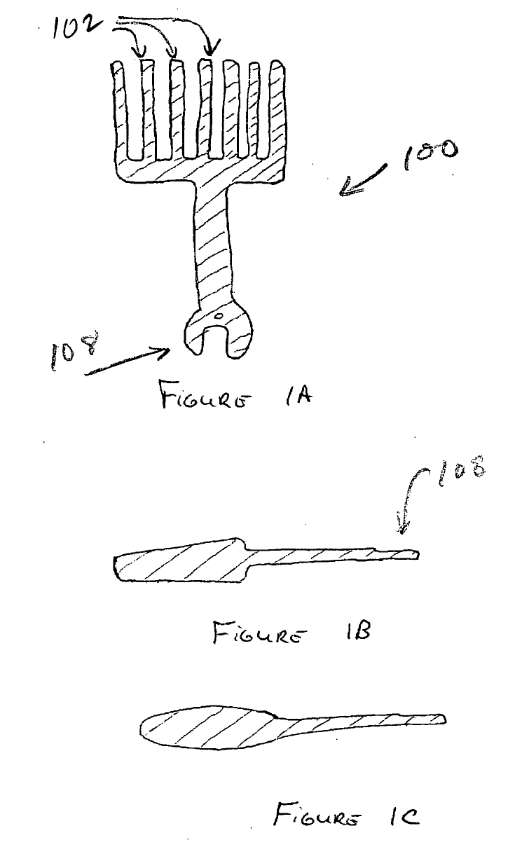

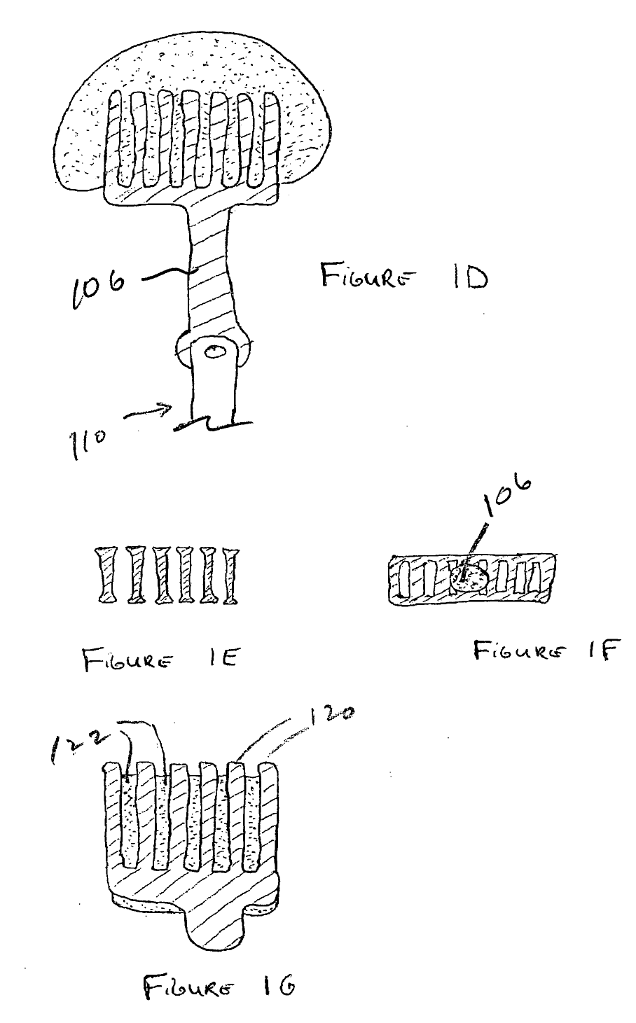

FIG. 1A is the view of the top of a disc evacuator according to the invention. The leading edge of the tool 100 has two or more comb-like projections 102. In the preferred embodiment of the invention, the trailing edge of the evacuator (C-shaped opening 108) fits into a power tool. A removal handle may be attached to the evacuator. The evacuator can be impacted into the disc space. Fluoroscopy or other navigational tools may be used to help align the evacuator with the disc space. The impaction handle may be removed after the evacuator is positioned within the disc. The evacuator could be connected to the power tool after the evacuator is positioned within the disc. Alternatively, the evacuator could be advanced into the disc under power.

The tool 100 oscillates from side to side in the preferred embodiment. The evacuator could also oscillate towards and away from the power tool, up and down, or in a combination of the above, such as a circular motion. However, in all of the embodiments described herein, instruments according to the invention may oscillate or vibrate in a left to right, cephalad to caudal, or anterior to posterior direction.

Alternative combinations of these and other motions may alternatively be used, depending upon the application and desired effect. For example, the tools may reciprocate. They may repeatedly rotate a few degrees (1-45 degrees) in a clockwise direction followed by rotation a few degrees in a counterclockwise direction. The tool may be driven by ultrasonic vibrations. The entire blade of the instrument may move uniformly. Alternatively, the tip of the blade may move through a greater range or arc of motion than the end of the blade that is attached to the power tool. The end of the blades that attach to the power tools may be configured to cooperate with current or future power tools.

FIG. 1B is a lateral view of the device drawn in FIG. 1A. The end of the evacuator that attaches to the power tool is drawn on the right. FIG. 1C is a lateral view of an alternative embodiment, wherein the top and bottom edges of the device are circular in shape. FIG. 1D is an axial cross section of the disc and the device drawn in FIG. 1A. The evacuator is connected to a power tool 110. The evacuator cuts disc and shaves the disc from the VEPs as the power tool moves the instrument. For example, the power tool could move the evacuator from side to side. The evacuator could be controlled by a computer directed machine, such as used in CNC machining.

FIG. 1E is a coronal cross section of the cutting portion of the evacuator drawn in FIG. 1A. FIG. 1F is a coronal cross section of the device drawn in FIG. 1A. The cross section was taken through the shaft 106 of the instrument. The holes between the cutting tools, at the back of the instrument, permit disc material to migrate out of the tool and the disc.

FIG. 1G is a view of an alternative embodiment wherein the evacuator uses a pair of cutting components 120, 122. In the preferred embodiment of the device the paired cutting components reciprocate towards and away from the power tool. The top and bottom of the blades may have teeth. This embodiment of the device is particularly suited to shape the VEPs. The device may be used to prepare the superior and inferior VEP simultaneously.

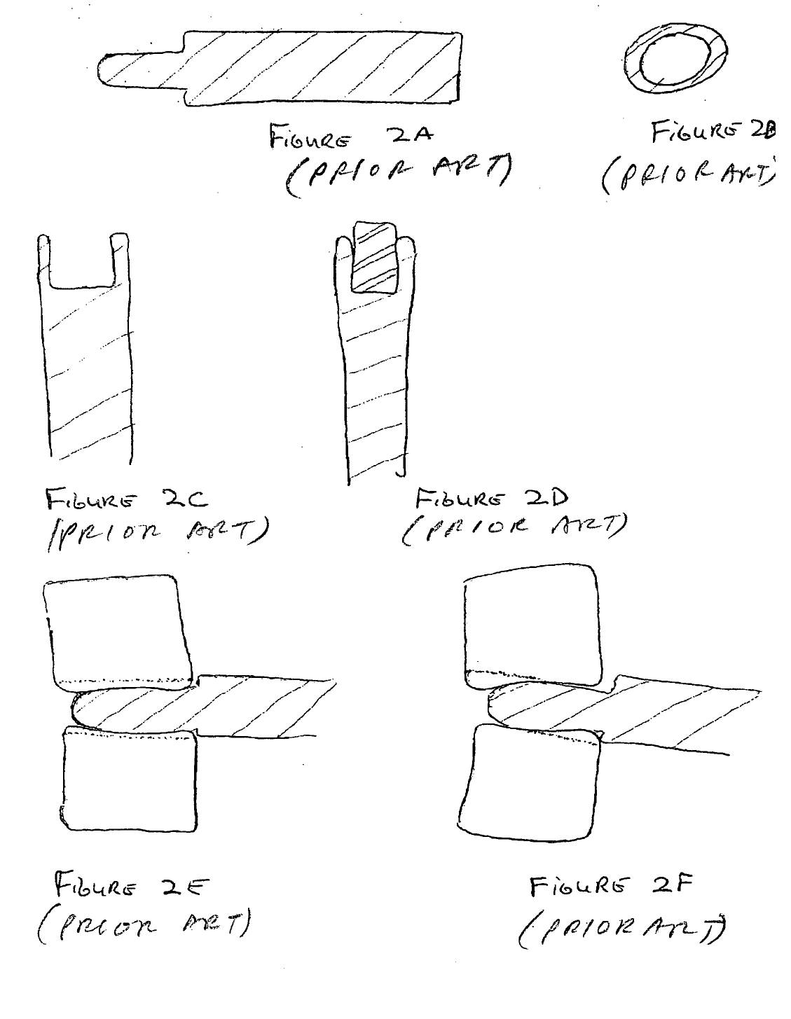

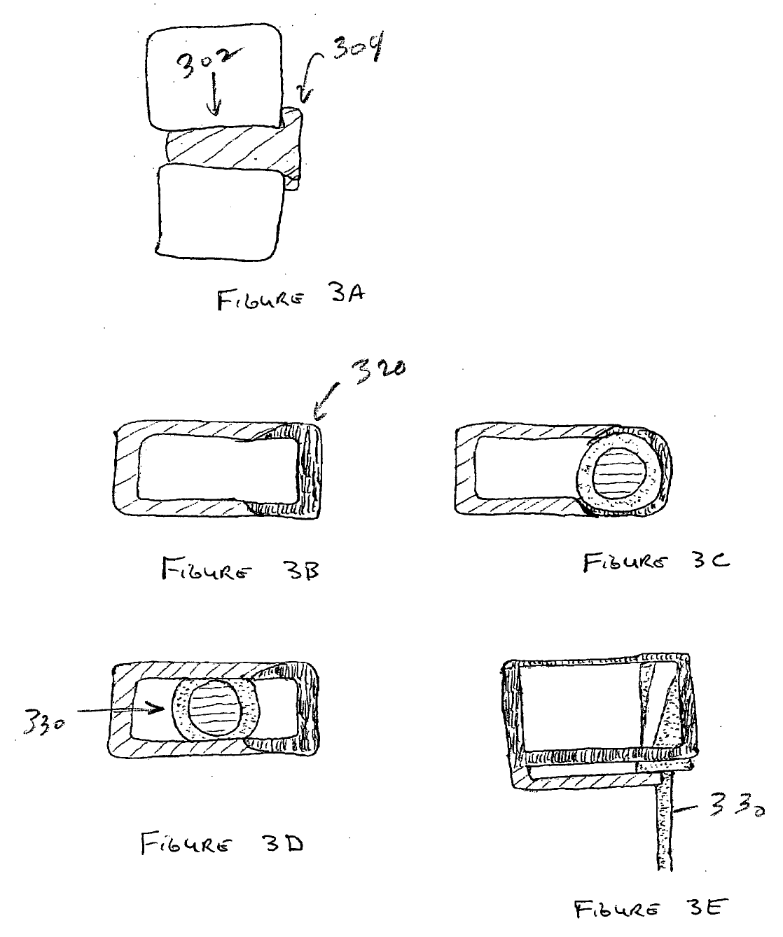

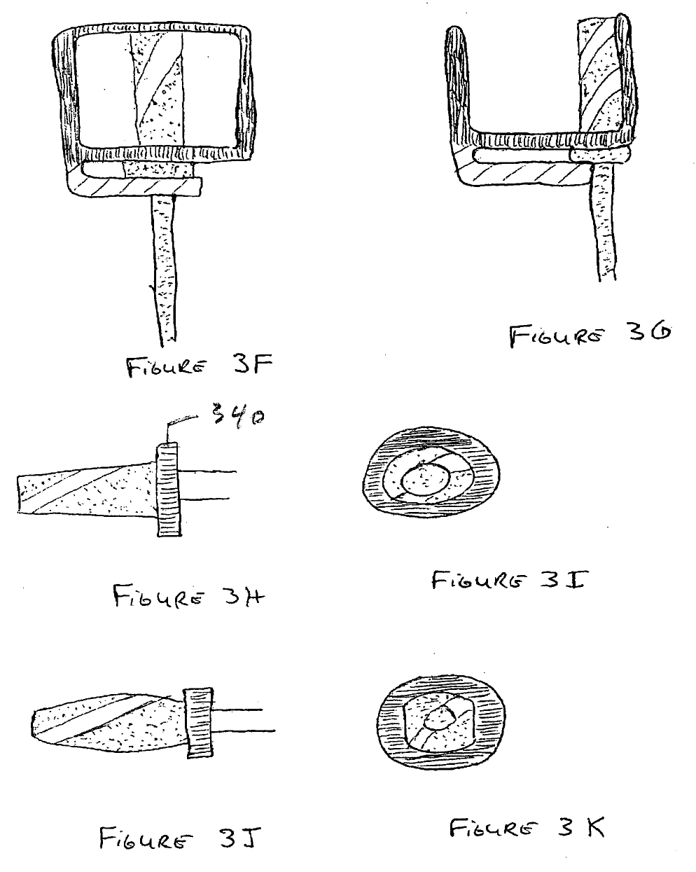

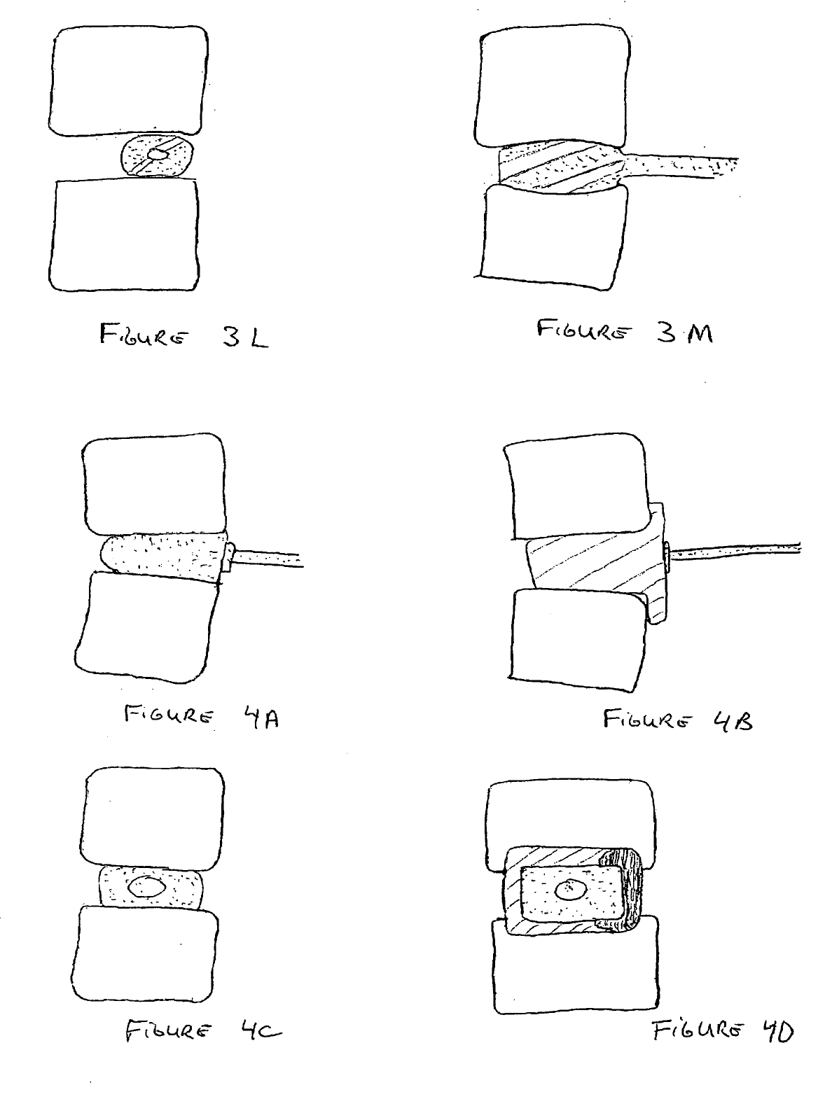

FIG. 3A is a lateral view of the spine and a novel milling guide. A first portion 302 of the guide fits into the disc space. A second portion 304 of the guide fits over the front of the spine. The guide may be impacted into the disc space with the aid of a removable handle. The guide improves upon the prior-art guide drawn in FIG. 2A in several important ways. First, the second portion of the guide that lies over the anterior aspect of the spine prevents tilting of the device. The leading end of the device that fits into the disc is less rounded than the leading edge of the prior art device drawn in FIG. 2A. Second, the rounded edge of the device drawn in FIG. 2A, facilitates tilting of the device. Tilting of the device leads the sub-optimal hole placement depicted in FIG. 2F. Third, the removable handle of the present invention improves the surgeon's view of the disc space. Surgeons are unable to see the disc space through the guide drawn in FIG. 2A, once the drill is inserted into the tube guide. Fourth, the novel guide permits surgeons to mill across the surface of the VEPs. The invention guides a rotating cutting tool from side to side across the surface of the VEPs. Thus, the invention guides cutting tools in an anterior to posterior direction and a left to right direction. The prior-art guide guides a cylindrical cutting tool (drill or reamer) in an anterior to posterior direction only. Both devices guide the cutting tool in a superior to inferior direction. As noted above, the novel guide controls the cutting tool in a superior to inferior direction better than the prior art cutting guide.

FIG. 3B is a view of the front of the device drawn in FIG. 3A. FIG. 3C is a view of the front of the cutting guide drawn in FIG. 3B and a cross section of a cutting tool. The device is designed to permit insertion of the cutting guide into the circular opening on the right side of the drawing (area 320). FIG. 3D is a view of the front of the device drawn in FIG. 3C and a cross section of a cutting tool 330. The cutting tool has been partially advanced across the front of the device from the left side of the device to the right side of the device.

FIG. 3E is a view of the top of the cutting drawn in FIG. 3A and the cutting tool. The Cutting tool has a circular projection at the trailing end of the cutting portion of the tool. The circular projection fits in a slot in the guide. The slot of the guide and the circular projection on the cutting tool cooperate to guide the cutting tool across the VEPs.

FIG. 3F is a view of the embodiment of the invention drawn in FIG. 3E. The cutting tool has been advanced across the guide. The embodiment of the invention may be used to remove disc material. The embodiment of the invention may also be used to shape the VEPs. The slots in the cutting tool are designed to push the loose disc material and bone from the disc space.

FIG. 3G is a view of an alternative embodiment of a cutting guide and the cutting tool. The open leading edge of the guide permits impaction of the guide into the disc. This embodiment facilitates use of the device for disc removal. Both embodiments of the guide could be used after distracting the disc space. The guides could be used over removal distracters that fit into the space for the cutting tool. Alternatively, the guides could incorporate a distraction mechanism. For example, the left and right sides of the guides could include scissor jacks.

FIG. 3H is a lateral view of the end of the cutting tool drawn in FIG. 3F. The circular guide is depicted at 340. FIG. 3I is a view of the end of the cutting tool drawn in FIG. 3H. The cutting tool is circular in cross section. The cutting tool is also tapered. The cutting tool may also have parallel cutting surfaces. FIG. 3J is a view of the end of an alternative embodiment of a cutting tool is designed to create domed shaped troughs across the VEPs.

FIG. 3K is view of the end of the cutting tool drawn in FIG. 3J. The tool has flat and rounded surfaces. The cutting tool is narrower as measured from flat surface to flat surface than the tool is as measured from rounded surface to rounded surface. The narrow cross section facilitates insertion of the tool into the disc space. The tool is inserted with the flat surfaces of the tool parallel to the VEPs. FIG. 3L is an anterior view of the spine and a cross section of the cutting tool drawn in FIG. 3K. The cutting tool has been inserted into the disc space with the flat surfaces of the tool parallel to the VEPs.

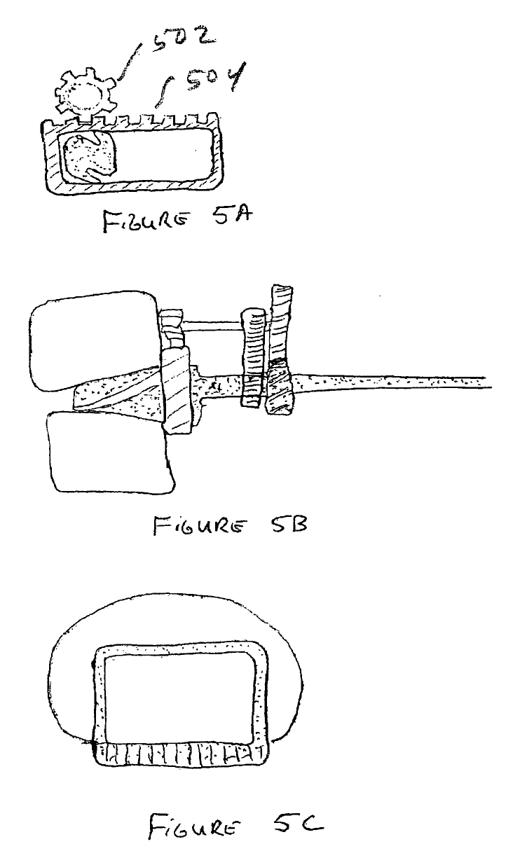

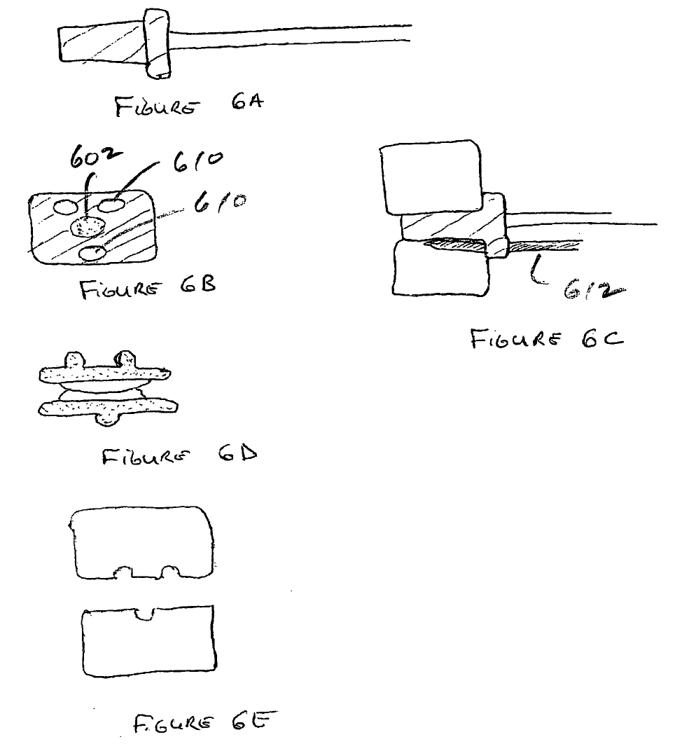

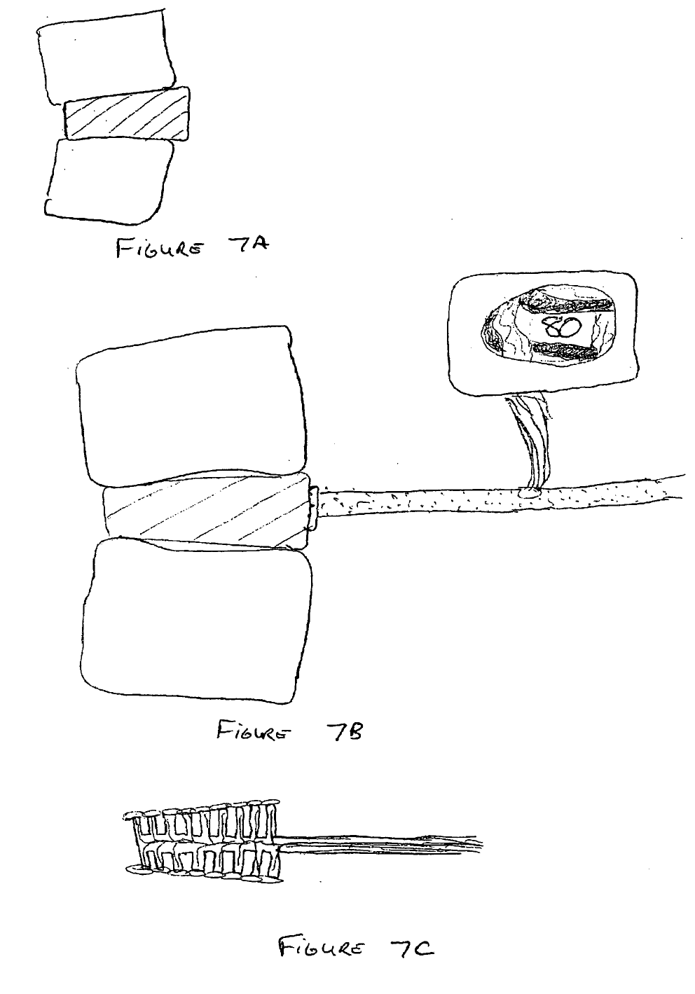

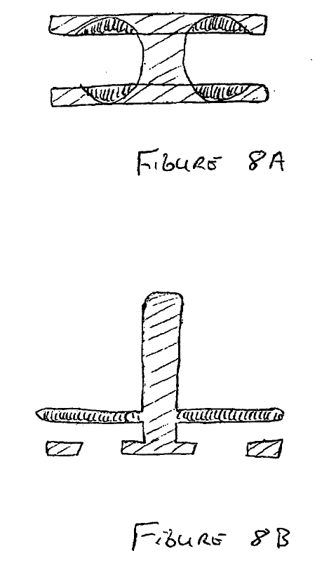

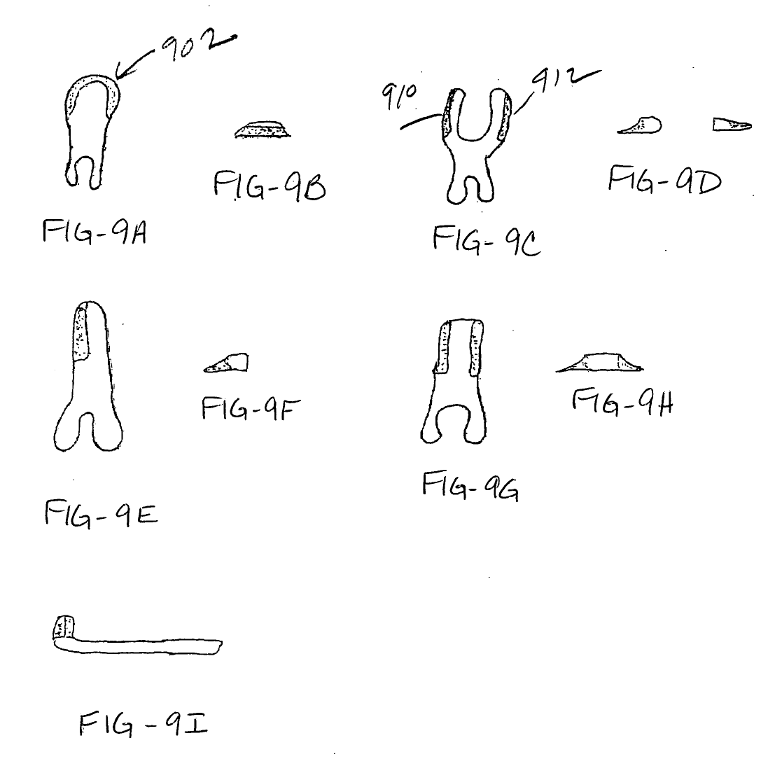

FIG. 3M is a lateral view of the spine and the cutting tool drawn in FIG. 3M. The cutting tool has been rotated 90 degrees relative to the position drawn in FIG. 3L. The rounded shape of the tool cuts rounded shapes in the vertebrae. FIG. 4A is a lateral view of the spine and a distraction device. The distraction device was impacted into the disc space. FIG. 4B is a lateral view of the spine, the distraction device, and the cutting guide drawn in FIG. 3G. The cutting guide was placed over the distraction device. FIG. 4C is an anterior view of the spine and the distraction device drawn in FIG. 4A.

FIG. 4D is an anterior view of the spine, the distraction device, with the cutting guide of FIG. 4B placed over the distraction device. The intradiscal arms of the cutting guide maintain distraction of the disc space after the distraction device is removed. In alternative embodiments of the invention, impaction of the cutting guide into the disc space distracts the vertebrae. The cutting guide may also contain a distraction apparatus, such as scissor jacks. The alternative embodiments of the device do not place the cutting guide over a distraction plug.

FIG. 5A is coronal cross section through an alternative embodiment of the invention wherein the guide and the shaft of the cutting tool utilize a mechanism to spin the cutting tool and to drive the cutting tool across the disc space. This embodiment eliminates the need for surgeons to apply lateral pressure on the cutting tool. The drawing depicts the use of gears 502 that cooperate with teeth 504 on the cutting guide and the shaft of the cutting tool. The drawing also illustrates on of many alternative shapes of the cutting tool. FIG. 5B is a lateral view of the spine and the embodiment of the invention drawn in FIG. 5A. FIG. 5C is an axial view of the disc and the embodiment of the cutting guide drawn in FIG. 5A. The teeth of the guide are illustrated by the vertical lines.

FIG. 6A is a lateral view of a novel distraction and drill guide. The distracter is impacted into the disc space. FIG. 6B is an anterior view of the distracter drawn in FIG. 6A. The removable shaft of the instrument is illustrated at 602. The circles such as 610 represent drill holes. FIG. 6C is a lateral view of the spine, the distracter drawn in FIG. 6A, and a drill 612. The holes in the guide direct drills into the vertebra above and below the disc space. FIG. 6D is an anterior view of an ADR with novel keels that fit into the holes created by the guide and drill drawn in FIG. 6C.

FIG. 6E is an anterior view of the spine. The guide of FIG. 6B was used to create holes that will receive the keels of the ADR drawn in FIG. 6D. FIG. 7A is a lateral view of the spine and a novel pressure transducer placed into the disc space after evacuating the disc and possibly after shaping the VEPs. The pressure transducer detects areas of the VEP that are not touching the transducer or areas that just touch the transducer. The device may be used to direct additional preparation of the VEPs. The transducer may be used after cutting bones in other areas of the body. For example, the transducer may used during knee, hip, ankle, shoulder, wrist, and elbow replacement.

FIG. 7B is a lateral view of the spine, the transducer drawn in FIG. 7A, and a monitor. Electrical signs from the transducer could be converted to numbers and a topographic image on the monitor. The monitor may use different colors. A number may displayed on the monitor. The number could indicate the percent of the transducer that has adequate contact with the machined bone. A microprocessor may assist with conversion of the electrical impulses. FIG. 7C is a sagittal cross section of the transducer drawn in FIG. 7A.

FIG. 8A is an anterior view of an alternative embodiment of the guide drawn in FIG. 3B. The guide depicted in FIG. 8A is placed into the space created after the use of the guide depicted in FIG. 3B. The circular openings on the left and right side of the guide accept the cutting tool of FIG. 3H. The guide is used to shape the VEPs lateral to the area allowed by the cutting guide drawn in FIG. 3B. FIG. 8B is an axial cross section of the guide drawn in FIG. 8A.

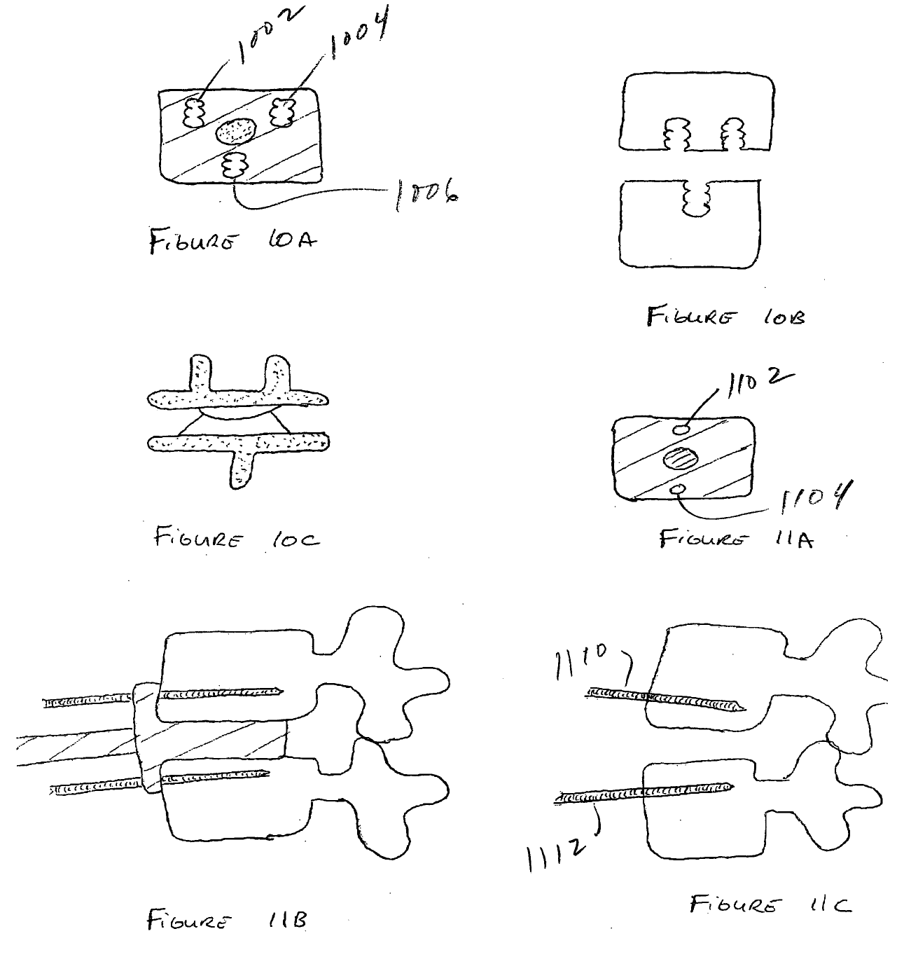

FIG. 9A is the view of the top of a blade designed for use with a power tool. For example, the blade could be attached to an oscillating power tool. The cutting edge of the tool is depicted at 902. Rapid oscillation of the cutting tool reduces the pressure surgeons must apply the tool. Prior art, non-power instruments such as curettes and elevators require a great deal of pressure to cut or separate the tissues. The reduced pressure required to operate the power tools decreases the risk of an instrument slipping if the resistance provided by the soft tissues drops suddenly.

FIG. 9B is a view of the end of the cutting tool drawn in FIG. 9A. FIG. 9C is a view of the top of an alternative cutting bit with cutting surfaces 910, 912 along the sides of the bit. FIG. 9D is a view of the end of the cutting tool drawn in FIG. 9C. FIG. 9E is a view of the top of an alternative cutting bit. The bit has a cutting surface along one side of the bit. FIG. 9F is a view of the end of the cutting tool drawn in FIG. 9E. FIG. 9G is a view of the top of an alternative cutting bit. FIG. 9H is a view of the end of the cutting tool drawn in FIG. 9G. FIG. 9I is a view of the side of an alternative cutting tool. The cutting portion of the bit is at angle to the shaft of the tool.

FIG. 10A is an anterior view of an alternative embodiment of the invention related to that drawn in FIG. 6B. The guide contains holes 1002, 1004, 1006 that allow multiple passes of a drill bit. The combined holes in the vertebrae prepare slots to receive the keels of an ADR or other intradiscal device. Drills are used to remove bone rather than prior art chisels. Chisels create a slot in the vertebrae. The fracture plan created by the chisels may propagate and result in fractures of the vertebra. Using drills decreases the risk of vertebral fracture. First, holes may be created with drills while applying little pressure to the vertebrae. Chisels are impacted into the vertebrae. Impaction of instruments injures bone around the slot created in the vertebra. Second, drills create cylindrical holes. Fractures are less likely to propagate through cylindrical holes than they are slots with flat or angled ends.

FIG. 10B is an anterior view of the spine after creating holes with the invention taught in FIG. 10A. FIG. 10C is an anterior view of an alternative embodiment of the invention drawn in FIG. 6D. The keels are longer than the keels of the ADR drawn in FIG. 6D. The ADR may be impacted into the slots created by the drill. Alternatively, a chisel may be used to connect the holes created by the drill. The holes in the guide drawn in FIG. 10A do not need to interconnect. A chisel may be used to connect holes in the vertebrae.

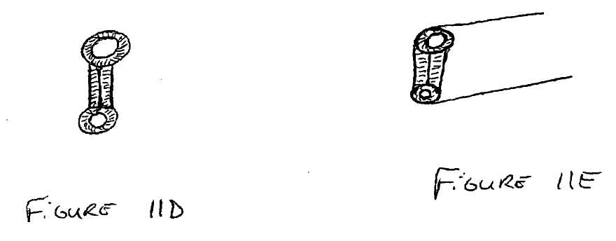

FIG. 11A is an anterior view of an alternative embodiment of the invention drawn in FIG. 10A. The guide has two holes 1102, 1104. Alternative embodiments of the invention may include one or three or more holes. FIG. 11B is a lateral view of the spine, the embodiment of the invention drawn in FIG. 11A, and two K-wires. The K-wires pass through the holes in the guide. The guide may also, but not necessarily, distract the disc space.

FIG. 11C is a lateral view of the spine and two K-wires 1110, 1112. The guide has been removed. Surgeons may check the position of the K-wires by Fluoroscopy or other imaging techniques. Navigation systems may be used to assist with the insertion of the K-wires. Surgeons may reposition the K-wires, if the locations or course of the K-wires are unacceptable. Drill bits may be used as an alternative to K-wires.

FIG. 11D is an end view of a cannulated chisel. The chisel is passed over the K-wires to create a slot for keels of an ADR. The chisel is passed after surgeons confirm the location of the K-wires. K-wires may be repositioned with little injury to the vertebrae. Repositioning chisels may result in substantial injury to the vertebrae. FIG. 11E is an oblique view of the embodiment of the invention drawn in FIG. 11D.

1. Apparatus directed to the efficient removal of disc material in conjunction with spinal surgery, comprising:
an evacuator having one or more prongs used to cut disc material and/or shave disc material from a vertebral endplate.

2. The apparatus of claim 1, wherein the evacuator is configured for attachment to a power tool.

3. The apparatus of claim 1, wherein the evacuator is controlled by a computer-directed navigation or other machine.

4. The apparatus of claim 1, wherein the evacuator includes a pair of plates, each with prongs, and either or both of which is moveable.

5. Apparatus directed to the efficient removal of disc material in conjunction with spinal surgery, comprising:
a guide configured for insertion into an intradiscal space; and
a cutter that fits into the guide for a controlled removal of disc material.

6. Apparatus directed to the efficient insertion of a disc replacement device having one or more keels, comprising:
a body having one or more guides to remove vertebral material corresponding to at least one of the keels.

7. The apparatus of claim 6, wherein the guide is a drill guide.
